SEQUENCE LISTING (43 sequences)

SEQ ID NO: 1 - DNA, 24 bp, Mus musculus
agcactctccagcctctcaccgca

SEQ ID NO: 2 - DNA, 12 bp, Mus musculus
gatctgcggtga

SEQ ID NO: 3 - DNA, 24 bp, Mus musculus
accgacgtcgactatccatgaaca

SEQ ID NO: 4 - DNA, 12 bp, Mus musculus
gatctgttcatg

SEQ ID NO: 5 - DNA, 24 bp, Mus musculus
aggcaactgtgctatccgagggaa

SEQ ID NO: 6 - DNA, 12 bp, Mus musculus
gatcttccctcg

SEQ ID NO: 7 - DNA, 1119 bp, Mus musculus
taaacaggctctcctctcacttatcaactgttgacacttgtgcgatctctgatggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccgtacatcgtcaaccgcacctttatgctggccaaggaggccagccttgcagataacaacacagacgtccggctcatcggggagaaactgttccgaggagtcagtgctaaagatcagtgctacctgatgaagcaggtgctcaacttcaccctggaagacgttctgctcccccagtcagacaggttccagccctacatgcaggaggtggtacctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtctgagcgagaagaagctagaaaacgaagaactgctccttcctgccttctaaaaagaacaataagatccctgaatggacttttttactaaaggaaagtgagaagctaacgtccatcatcattagaagatttcacatgaaacctggctcagttgaaaaagaaaatagtgtcaagttgtccatgagaccagaggtagacttgataaccacaaagattcattgacaatattttattgtcactgatgatacaacagaaaaataatgtactttaaaaaattgtttgaaaggaggttacctctcattcctttagaaaaaaagcttatgtaacttcatttccatatccaatattttatatatgtaagtttatttattataagtatacattttatttatgtcagtttattaatatggatttatttatagaaacattatctgctattgatatttagtataaggcaaataatatttatgacaataactatggaaacaagatatcttaggctttaataaacacatggatatcataaaaaaaaaa

SEQ ID NO: 8 - DNA, 7445 bp, Mus musculus
gtctatcacttgcttaagattcttctaatttataaaaaaaactatttcttaaaatgaaaagcaaacagagcacgtatttatagcatggtgttctgaccatgcaggtacagagtggaatggtaagaggcgctattatcagcattaaccaacatgttaatgttttcttctggcaagcaaacttgaaatctatgtcttaaacaatcttcaagcctctaatatagtgctaacgactggagtccgctgctgtccaacagagctcttgagcacgctctcctctgtttgcaattttatgttctttgatcgactccccaacctctcaccttcggctcctgatggccacctttcaactttctgcatttatgaactccatgttttaatctttttattaaaatattcacacaatcagtgtttgtgcaagtctgtttcacccacatgtatgtctgtgcaccaagtgctgcctggtgcttgtgggggcaaggagcaggagagggtgccctggcaccggagtcacggatggttgtgagccaccatgaggatgctgggagttagacccaggtcctccagaagtgcagcaaatgctcttaaccacacgcaggcatttctctctccagccccaacatgagtgcttttagattccacctagaatagagatctgatggcttcactcactgccacctcccctttgcatctttctgccaaggaacaccaaaaagcaagaatccccacactgctttcgctcctcaagtctgcacctctcaacaggtcaagattctccagtgtccctctaacactttccccagtgtccctctaacactttctccagtgtccctctaacactttctccagtgtccctctaacacttttgatctcaattagctgaggggagaaagatctcacacagtgattttcatgacttcgcgttctagtctagatgtaggcatttgcgtgtcagtctagggtaggcgtctgctcccgctgcttaggaaagactttcctagtctagttgtcaggtgctatctgggattcagtgtacatacaatgcaaaaaatcccagtattttgtaaattctcttcttcaactatccatctatatagtatgttattgtaggctcatttaaaaataatattttgagacttatgcttgcacaagtaaaatgtcagagaattagcaaatgtatagtattattttattttaaaaaaatctatgcttaaaatgtctattagattgttcactaccgatatttccaaacttaacttgaccttggctatgatttcaacctttgtatttgcatctaccataacagtctctgaaccagaacattctgtggcaatgggagctgtgaagaaagccaacattcttattaaaaaaaaaaaacagctagttatagtttaggattccatatactaaaaaaaatagagatataattattttaaaaattgaaataatctccaagttttcattatggcttatttcaaagcacagaatataggacacgggtcttttatttctggtcacttctaaagagataagaatctatgaagttggtgggaaaatgagtccgtgaccaaaacgctgactcaatagctacgggagatcaaaggctgctctactcaatcagaatctactacggcaaagccatggctttctttgaaaaccgtgtttagaagatttctgggatttgtgtgcaaaagcaccttgttggccctcaccgtgacgttttagggaagacttcccatctctcaaggtgggaaggcttggaggtggtgtcttgtggcctcctatggtggttaggtacttctcagaagacaggactggaaattagataatgtctgatgtcatatcattcacaataccaaaaaaaccctggtgtcccgatggctataaaagcagcaacttctgcctctcccatcacaagcagagacacctaaacaggtaagcactcagacctctacagacaatcatctgcttggtaccatgctacccgacgaacatgctcccctgatgtttttgccttttgctctctcactaacaggctctcctctcacttatcaactgttgacacttgtgcgatctctgatggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccgtacatcgtcaaccgcacctttatgctggccaaggaggtacagctgcatctctttctctccataccgccttgccattttctctgaagcacttgcaaactctttaggggcgctttatctccgcaggtctcactacctatgttttctgtctctttagagactctttaaggactgggtctttttctatttctatttcaaggtctcaggaccatttcctatcttggccttcaggacacatatactgaattttatctacagaggcgcatttagaaagccacccacgactgcaatactttccatttctctgtgctctcttctgaactcatactctcttggctactcctgagacccactgcggacatacatctctacttacaggcttttcttccatctccttgtcacccaggcacttagggttttctctctttcaggccagccttgcagataacaacacagacgtccggctcatcggggagaaactgttccgaggagtcagtgtaagtcctcactgtgatgagcagggctagctgcgggagctggtggaccctctgggatagtctgacgtatgacccctgctgcttcttgtctacctgcaggctaaagatcagtgctacctgatgaagcaggtgctcaacttcaccctggaagacgttctgctcccccagtcagacaggttccagccctacatgcaggaggtggtacctttcctgaccaaactcagcaatcagctcagctcctgtgtaagtctgactctggctacctatgctcctctctcttcctcttctattccagtaagaacccgaggtcctgccctctctctcttcacaagagtgaggagggcctcagcaccaccaccatcataggccacttgaaataggtcacaaaggctttggcttcaattgagtaatactttgagtttgtatgagtgaagctttatttgttttatccatggaaagaaatcaactcaaattctgtaggatgagaaagatgttgggaacgaaaaaaggcctagatagagaaacagatctgctgagtatagtacttatggggggagcagggggcgatatccactgagtacaagtacttgtggggagagaaatccactgagtacaagtacttgttggcatggagatccactgagtacaagtacttgtggggggagggaatggcacagagcaaaagttgaagggaaggaagatggagaggcctcatggttgggggtgtgaaaggtcactccttttccatgtgatggagagttaagaaaaaccagtgtgtgagtttgatgtcttcagacacccccaactatgaaacatatccacgaggagcgggcagactgtgggagacctggcatttagggaaggcgcggcttttcacacgagaaactttatgctcatctcttgtgctacactcccacctttgatgaggttcagctcaggtttcgtttctaccgttcttgctactggtggaaacttcagtaggattccccaaagacgaggacagctcttctgtaagggagggacctggatttcagtgtcctagagaacgaaatagctcagagaatctaggtcaacgtgaaatctaggtcacagcgggcaaaaatgactgaacgcctctattccaggtgaacggtcacgtgcctcagatatactgaggtattgggctcccaccggataagattctgttagtgagtctgcttttattttgcagcacatcagcggtgacgaccagaacatccagaagaatgtcagaaggctgaaggagacagtgaaaaaggtactattggcaagccacaatactaagccattcagtaggagacgtggggatttctttctctgcttcccagtcccttctactttgtaacattttatttgacttgtctactatctggtccattactcgcttagctgcacctgtatctagctgggtctatagatctttcaatctgtgtctaaatttgtaagtcacaattctggagctagcagaaagcttagctcagccagtctcatgagcacttgctcggaggatggcttgtgacagagtcaatgctagaagacagcatccctgattcccagctctgcacttgcctagtggccatgtgtaattactttggcttgattaagtatttgggaaagccagttcccacggacctacataatctgaagaaccatgcattgaaaactagaaagctgggcacaaacttactagagatgatttttgagctcattaaacggatgctctgaaatgtggcaaaatcaacccagaataacaacaaaagagctggatttgcaaataggacaagtatttagaatcactggtattaatagctatcatcttaattaaaatatagggcctatatatatatttaagattaaacacaagagtggatagcctcccaatttacttggcctggtttcaaaagagtaaaaatatcagtcatggattaattatagtgtcatgaaagtatgagatggaaaccctttccttactttttaccttcatttcttagttttttttttcttcacaccctgatcaagccactagtaagcacctatctgctgtgagctattatatgactttacagcaaacaacattgctgtgtggcctctttggggaagggaacaggatagcaggaggctcaggctagcaagtctgacttgccctaaagccagaggcatggttgatagcagagaaagtgaggctcttcgcaagtgggtgtgcttaagtaatcagaaacaggaaggctccggttgatggaattatcagtaagatatctacccttatctccttctatcgaacctaaatcgtctctttttcttgtgtgtaggctgataaacacacttgttttcttttgagtgttcatggctttgtagatttttagtgctctgccagttcttgttagagggtttgttaccttgacacctgggcttggatgttagcatgccaaaggcacacacttctgaatgcctgtgtaaaaggttattattcatttactttgtctttggaaaggtgaagcgtgtgtgagaaagaactcacaggagatgtgttctctgtaggaaaactttttttttccccttaaatgcctataatccactttcagtcaactttgacttttataccatgctgtcacatgaaagagtgtttaggcccgctctcatggctctgggaaaagcaccaataggggaaggaatgttatgctgagaaatctgaccggcagggaaactggtcagagctcccccgaagaccaccacaggtgttaagtaggaacagtccagggtgggctcatgtaatagaatggaacagagcgagggaagataagctacaaagtttcatagggtccggagtcttaaagatacaaaatagctgcttgggcttcataacaaaggaagtctgggaaggcagcaagtgagagggaaatggaaagggaaaaaacagaatgtagaggacttgaacagctacaaatcctctaccagacgatttttcttggaacaatctagaaggtagtggattaggtgattgcagggggacttgctttgccatttgaatctgggtttttgtctctccattgaggttgaaagcgtcaccctttttaccctcgaatggaggaggaaagaaggggtgttatgactcctacctggagttttactagtttacgcaatggaacagacactcgggacctcctcttgacaaaaaaaatggaaacctgttgtttgtcttgtttgttcttttgttaagaaagcacaggcaaagcccgaccacatgggttgaatgtgggtctttgagtcaaggcttttgagttgagcactcatcaatagttgatcatggtcaggtggagggctacctgtcaggccgagccctgctggcttcgcacttaacatctccaggtctcagtatcacttcctgctacttagcacagttaggagttgagcaaacctttttttccaacccccactaaaatttaattgacaaaagactgtgtaatttgtgggatacagtgtgataattgatctatgtgtgcattgtgcaaggttcaataagatagattaataggcccatcaacagctttatgggtgtgaaatgcaagtaatataggtagatgcctgtggtgtccttaggtcagaaaggcatgattttaaggtcttgggcaaatcatattatactcatgctaaaaatacattatgttgattattaatcttttagagaaggctgatacttggttttggtgctcagcaagcaaatgtcaccagctctttctaactggtaccactttagaaaatgctacctgtgctcaaattggtttgtattcttattttcatagcttggagagagtggagagatcaaggcgattggggaactggacctgctgtttatgtctctgagaaatgcttgcgtctgagcgagaagaagctagaaaacgaagaactgctccttcctgccttctaaaaagaacaataagatccctgaatggacttttttactaaaggaaagtgagaagctaacgtccatcatcattagaagatttcacatgaaacctggctcagttgaaaaagaaaatagtgtcaagttgtccatgagaccagaggtagacttgataaccacaaagattcattgacaatattttattgtcactgatgatacaacagaaaaataatgtactttaaaaaattgtttgaaaggaggttacctctcattcctttagaaaaaaagcttatgtaacttcatttccatatccaatattttatatatgtaagtttatttattataagtatacattttatttatgtcagtttattaatatggatttatttatagaaacattatctgctattgatatttagtataaggcaaataatatttatgacaataactatggaaacaagatatcttaggctttaataaacacatggatatcataaatcttctgtcttgtaatttttctccctttaatatcaacaataccatcatcatcatcattacccaatcattctcatgatttcatgcttgacccatattatactgttaaagttggttcctggaggcctgtggttttgtgtgtgttgtgtgtgtgtgtggggttatgcatgtgaaagccagagatggatattaggtgttcttctctatcagtctttgccttattatttgagacagggtctgtcactgaacctgtagctaggctggccaacaagctctattaattttttttaagattaattaattatgtgtat

SEQ ID NO: 9 - DNA, 1111 bp, Mus musculus
aacaggctctcctctcagttatcaacttttgacacttgtgcgatcggtgatggctgtcctgcagaaatctatgagtttttcccttatggggactttggccgccagctgcctgcttctcattgccctgtgggcccaggaggcaaatgcgctgcccatcaacacccggtgcaagcttgaggtgtccaacttccagcagccgtacatcgtcaaccgcacctttatgctggccaaggaggccagccttgcagataacaacacagacgtccggctcatcggggagaaactgttccgaggagtcagtgctaaggatcagtgctacctgatgaagcaggtgctcaacttcaccctggaagacattctgctcccccagtcagacaggttccggccctacatgcaggaggtggtgcctttcctgaccaaactcagcaatcagctcagctcctgtcacatcagtggtgacgaccagaacatccagaagaatgtcagaaggctgaaggagacagtgaaaaagcttggagagagcggagagatcaaagcgatcggggaactggacctgctgtttatgtctctgagaaatgcttgcgtctgagcgagaagaagctagaaaacgaagaactgctccttcctgccttctaaaaagaacaataagatccctgaatggacttttttactaaaggaaagtgagaagctaacgtccaccatcattagaagatttcacatgaaacctggctcagttgaaagagaaaatagtgtcaagttgtccatgagaccagaggtagacttgataaccacaaagattcattgacaatattttattgtcattgataatgcaacagaaaaagtatgtactttaaaaaattgtttgaaaggaggttacctctcattcctctagaagaaaagcctatgtaacttcatttccataaccaatactttatatatgtaagtttatttattataagtatacattttatttatgtcagtttattaatatggatttatttatagaaaaattatctgatgttgatatttgagtataaagcaaataatatttatgataataactatagaaacaagatatcttaggctttaataaacacatgaatatcataaa

SEQ ID NO: 10 - DNA, 21 bp, Mus musculus
ctgcctgcttctcattgccct

SEQ ID NO: 11 - DNA, 21 bp, Mus musculus
caagtctacctctggtctcat

SEQ ID NO: 12 - DNA, 20 bp, Mus musculus
gacgcaagcatttctcagag

SEQ ID NO: 13 - DNA, 16 bp, Homo sapiens
atgtatttcccagaaa

SEQ ID NO: 14 - DNA, 17 bp, Homo sapiens
ccttttctgggaaatac

SEQ ID NO: 15 - DNA, 22 bp, Mus musculus
aggtgctcaacttcaccctgga

SEQ ID NO: 16 - DNA, 22 bp, Mus musculus
ccactctctccaagctttttca

SEQ ID NO: 17 - DNA, 21 bp, Mus musculus
caagtctacctctggtctcat

SEQ ID NO: 18 - DNA, 418 bp, Homo sapiens
agaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgttt

SEQ ID NO: 19 - DNA, 21 bp, Homo sapiens
tggccaggaagggcaccacct

SEQ ID NO: 20 - DNA, 21 bp, Homo sapiens
cctatcagattgagggaacag

SEQ ID NO: 21 - DNA, 36 bp, artificial sequence
  primer 24,25,29,30,34,35: n is inosine in all cases
ggccacgcgtcgactagtacgggnngggnngggnng

SEQ ID NO: 22 - DNA, 20 bp, artificial sequence
ggccacgcgtcgactagtac

SEQ ID NO: 23 - DNA, 20 bp, Homo sapiens
ccttccccagtcaccagttg

SEQ ID NO: 24 - DNA, 20 bp, Homo sapiens
taattgttattcttagcagg

SEQ ID NO: 25 - DNA, 690 bp, Homo sapiens
tgcacaagcagaatcttcagaacaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttt

SEQ ID NO: 26 - DNA, 4797 bp, Homo sapiens
tgcacaagcagaatcttcagaacaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggtatacatctcaatcctgctctttctcgttggatctacttggaatccaaatagttcttaaacttttcttcagagcatctctaagagctttaggaacccactgtttatccctgagggtagataaattttctgttttttcagagactctttgggaatctggctttttttttttcttgaacttcttccttccattttggcctttatgatacatatgatgaatttttcccaaagagcggccattcagtaatccatctgatgatttttttttcctttatgcctctgtgcattgttctaaactcatgcacacatctgaattctgcttttagtctttatgatgttgctctggggagacgggatggggcacatgtctatgtataaattttttttctatttgctcaatgtccagacccttagtcttttcttctcttccaggctagcttggctgataacaacacagacgttcgtctcattggggagaaactgttccacggagtcagtgtaagctacagttgtgacgaacagggccgtgtgccgtccatgggtacttggggtggtggtgatgatggtttaggtcttatcccttatgaccctttctgtttcccttccacctgcagatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtgtaagttcagctctcagcctatgcccacctacccctccttccctccttccacagagacccccttaccccaactctctctccttccccctacccctaagctagcaggaagaagtgtcttggcagcagtgttatcaggagtcatttgggatcatagagtatttgcttttgctttgactgagtcacatcttgagtttatagtggtgaatggggtctggaacttaagtgtacagaagccgcattggtttgtcttcggaaaaaaggcaactcaggttgcgtaagatgagaaaggtgttgggaaaacatctagctgtggaaatggatccattgagtctaagttgttgaggggaggggatggcatggagagaaattagaagagaaagtgggaaatgggaaggcttaaagtcggtggtgggtcggcagactgttgccctgttgatgtcatgggaagccacaaaatcggaggcgtgtgaacttgatgccgctgaacatttgaaactatgaaaaaaagtttgagtggagtgggcccagtaaaaggccctaggacttactgaagagggcttaattttcacatgagatgttttatgtacatttcttgttctaagcatgcaattttctggagatacgattgaggttttattccttacagaatttgcataaactactccgctctttccacaaatgcaaacctcagtaggatttcccaaagatgaagagaggtctcttgtaagggaagtgactggattctggcgtccaagggaattcaagagctcaggaaatctaggtcactgttgaaatctaggtcattgtgggcaaaattactaagagctttaattccaggtgaattgtactgtacctccatgggtgtggaggttcataaagtttcagcacaacattaagatagttatgcttgttattgttttatagcatattgaaggtgatgacctgcatatccagaggaatgtgcaaaagctgaaggacacagtgaaaaaggtaggactgataactgtcaatgctaagtcatgcaataggagagacaaatgttgtttttctttcctttctttcttcccatcactttgtgatttttcacttgattctcctaccaccagggcgattactttggtgtctgtgtatgtagatatatctatatatctagatgtcagtttccaaatcttgcaaattgtagaattctagaactggttgggatcttagcttgtctagtcacataacctcagattctggggatggtcagtggcagagatagggctagaatgcaggtctcctgaatcccaagccagcacttttcccggtggtgatacagattagttttggtaccattaattcttagggaaatttcagattcctattgactcatgtaatctgaagaagtacttgtttaaaaacagaaaaatgcctatgggcaaatttatttgaagtcatttttgaagtcattaatgcattgctttgaaacttggaagaataaactcagaacaatgagaaaagagctggacttgcatatagggctaatttctggagtaataaacacttattttgaattatcataatatctatcagatattgattatagtttaaaagcaagagcagacaaccccgatctcttttatacaggttcaaatagagtaaaaatattagtaagagatttattatagttaaatggaagtctgaattggtaagcttttttttcttcctctctcccatcaagaccttccattctagtttcttccttcactccctcaacaaatccctagggagcatttatccatggtgggctggtgtacatttctatagtgaatgataccatcatgtggcctatttggtgaaaagaacaacaatggaaggcttagactaacaatagtgactcaccccaaaaccggaggaatgattaggagcagtgaaagtgacgctcttgcaagcaggtacaactaaatactcagaaacatgaaggctccagttgatggaattttcagtaacaagcttaaccttaattccccctttttccctcttgactttttaaaaaagcgtttcttcctgagcatcatttaatgagtgtgactgtttcttcctttgataattgaaggctttgtagttttaaattgtgaagcccagttctcttgttatagaactattatctagacatggagggctgaatgttagcatgccacagacaaggcatgctttacacatcttgcttaaaaaattactgatttcatcttgcttgttgtctttagaaaagtgaagtgtgagagaggagaatctcatggtgatctgtgtgattttcaagacctttaatccattttgaaagaatcaatttcatatttgcaatgggttgccatgtggaagagtgattatgcttttttgctggtagcttcagaaagcacaggagggagagcaatgttgttcagagaaagatcaacaggaggagaaactgtcagagctgtctgaaatagggtggttttgggaggcattaattccctctcgttgggggtaaaagcagaacgcaggttggtagtaaaatgcatgacagacagtaggggacgataaactttaaaattctttatagtcttggagtctttgagatagaaaagaatatctttttggccttatgtcaaaagaagtatggaaaggtgaaagggcggaagaaagcaggaaaaggaagaaccatgtattatatagaggacaatggtgacaaggtttttcttgaaataatgcaaatatgatagattagaggaatttcagtagggaatgcttttcacttgaatttgggtttcctcttcgattaagtttgggatcctcatctgcatttgacttggagagagaaagaatgaatgttaggacctatatctggttttctattaactaaagcaagtggaaaagacttatttggtatttttcccacaaaagtgaaaacttttcttttactgtttgtcaaaaaggtggaaatagaaaaagccttaatgtattggtgaatacatggttcaaagtcatttgagtagagatgttttaaatcaggagtgtccaatcatttggcttccctggaccaccttgaaagaattgtcttggtacacacataaaatacaagaacaatagctgatgagctaaaaaagtccatgcataaatctcatactgttttaagaaagtttatgaatttctgttagggtgcattcaaagctgtcctgggccatgtgcggcctgtgggctgcaggttggacaagctccttataagtaatctgtcatagatagttttggagctgcaaaacaggccaaggcataatgggtggcactcgggatcccccagatcccagcctcacttcagtctccttgctctggttaagaaggggtggtcaactctctgcccagcttttaaacagcttcattagtgtgaggtgcacctgaaattgatgcctgctggtggcctctcagtccagagagccgtcattttaagctctttggcaaatcatacaatactaaagggatattactatgaatgttttacaaatgcttaaaactcggtttctgtctccatcaacctaatcttgcaatttctaatttgttcactttagaaaacatggcataaatgctcaaatacttttgcattcttattttcacagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttt

SEQ ID NO: 27 - DNA, 20 bp, Homo sapiens
atcagatggattactgaatg

SEQ ID NO: 28 - DNA, 20 bp, Homo sapiens
agctcagctacagcacagat

SEQ ID NO: 29 - DNA, 20 bp, Homo sapiens
cctgccccatttattggcag

SEQ ID NO: 30 - DNA, 20 bp, Homo sapiens
tgtcctctgccaccctaaca

SEQ ID NO: 31 - DNA, 20 bp, Homo sapiens
taattcaccaggaccatcat

SEQ ID NO: 32 - DNA, 20 bp, Homo sapiens
gtggactcaggcaatgatgt

SEQ ID NO: 33 - DNA, 20 bp, Homo sapiens
acatagagtgttaaagtggg

SEQ ID NO: 34 - DNA, 20 bp, Homo sapiens
gctggaaggtggacagcgag

SEQ ID NO: 35 - DNA, 20 bp, Homo sapiens
tggcatcgtgatggactccg

SEQ ID NO: 36 - DNA, 20 bp, Mus musculus
tctgctccctgctcctggga

SEQ ID NO: 37 - DNA, 20 bp, Mus musculus
tccaggaggtctgtagtaat

SEQ ID NO: 38 - DNA, 21 bp, Mus musculus
ctgcctgcttctcattgccct

SEQ ID NO: 39 - DNA, 21 bp, Mus musculus
caagtctacctctggtctcat

SEQ ID NO: 40 - protein, 179 aa, Mus musculus
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 41 - protein, 179 aa, Mus musculus
MAVLQKSMSFSLMGTLAASCLLLIALWAQEANALPINTRCKLEVSNFQQPYIVNRTFMLAKEASLADNNTDVRLIGEKLFRGVSAKDQCYLMKQVLNFTLEDILLPQSDRFRPYMQEVVPFLTKLSNQLSSCHISGDDQNIQKNVRRLKETVKKLGESGEIKAIGELDLLFMSLRNACV

SEQ ID NO: 42 - DNA, 5935 bp, Mus musculus
gaattcaagtccacatgcaatcaatccgaatactttgtaaattctcttcttcaaatatccatctatatagtataagttattgtaggatcatttaaaaataatgttttgagacttatgtttgcacaagtaaaatgtcagagagaattagcaaatgtatagtattattttattttaaaaaatctatgcttaaaatgtctattagattgttcactactgacatttccaaacttaacttgaccttggctatgatttcaacctttgtatttgcatctaccataactgtgtgctcacttaccatgctatccgacgagcatgttcccctgatgtttttgccttttgctctctcgctaacaggctctcctctcagttatcaacttttgacacttgtgcgatcggtgatggctgtcctgcagaaatctatgagtttttcccttatggggactttggccgccagctgcctgcttctcattgccctgtgggcccaggaggcaaatgcgctgcccatcaacacccggtgcaagcttgaggtgtccaacttccagcagccgtacatcgtcaaccgcacctttatgctggccaaggaggtacagctgcatctctttctctccataccgccttgccatttctctgaagcacttgcaaactctttaggggcgctttatctccgcaggtctcactacctatgttttctgtctctttagagactctttaaggactggatctttttctatttctatttcaaggtctcaggaccatttcctatcttggccttcaggacacatatactgaattttatctacagaggcgcgtttagaaagccacccacgactgcaatactttccatcctgttgtgctctcttctgaactcatactctcttggctactcctgagacccactgcggacatacatctctacttacaggcttttcttccatctccttgtcacccaggcacttagggttttctctctttcaggccagccttgcagataacaacacagacgtccggctcatcggggagaaactgttccgaggagtcagtgtaagtcctcactgtgatgagcagggctagctgcgggagctggtggaccctctgggatagtctgacgtatgacccctgctgcttcttgtctacctgcaggctaaggatcagtgctacctgatgaagcaggtgctcaacttcaccctggaagacattctgctcccccagtcagacaggttccggccctacatgcaggaggtggtgcctttcctgaccaaactcagcaatcagctcagctcctgtgtaagtctggctctggctacctatgctcctctctcttcctcttctattccagtaagaacccgaggtcctgccctctctctcttcacaagagtgaggagggcctcagcaccaccaccatcataggccacttgaaataggtcacaaaggctttggcttcaattgagtaatactttgagtttgtattagttaagctttatttgttttatccatggaaagaaatcaactcaaattctgtaggatgagaaagatgttgggaacgaaaaaaggcctagatagagaaacagatctgctgagtacagtacttatggggggggggggcagggggcgatatccactgagtccaagtacttgttgggagagaaatccactgagtacaagtacttgtgggggaaggaatggcacagagcaaaagttgaagggaaagaggaagatggagaggcctcaatgttgggggtgtgaaaggtcactcctttttccatgtgatggagagttaagaaaaatcagtgtgtgagtttgatgtcttcagacaccccaactatggcagactgtgggagacctggcatttagggaaggcgcggcttttcacacgagaaactttatgctcatctcttgtgctacactcccacctttgatgaggttaagctcaggtttcgtttctaccgttcttgctactggtggaaacttcagtaggattccccaaagacgaggacagctcttctgtaagggagggacctggatttcagtgtcctagagaacgaaatagctcagagaatctaggtcaacgtgaaatctaggtcacagcgggcaaaaatgactgaacgcctctattccaggtgaacggtcacgtgcctcagatatactgaggtattgggctcccaccggataagattctgttagtgagtctgcttttattttgcagcacatcagtggtgacgaccagaacatccagaagaatgtcagaaggctgaaggagacagtgaaaaaggtactattggcaagccacaatactaagccattcagtaggagacgtggggatttctttctctgcttcccagtctcttctactttgtaacattttctttgacttgtctactgtctggtccattactcacttagctgcacctgcatctagctgggtctatagatctttcaatctgtgtctaaatttgtaagtcacaattctggagctagcagaaagcttagctcagccagtctcatgagcacttgctcggaggatggcttgtgacagagtcaatgctagaagacagcatccctgattcccagctctgcacttgcctagtggccacgtgtaattactttagcctgattaagtatttgggaaagccaattcccaccgacctacataatccgaagaagcatgcattgaaaactagaaagctgggcacaaacttactagagatgatttttgagctcattaaactgatgctctgaaatgtgatcaaatcaacccagaataacaacaaaagagctggatttgcaaataggacaagtatttagaatcactggtattaacagctgtcatcttaattaaaatatagtgtctatttagctgcctatttaagattaaacacaagagtggataacttcccaatttactgggcctggtttcaatagagtaaaaatatcagtcatagattaattatagtgtcatgaaagtatgagttggaaaccctttccttactttttaccttcatttcttagttattatttttttttcttcacaccctgatcaagccactagtaagcacctatctgctgcgagctattatatgactttacagcaaacaacattgctgtgtggcctctttggggaagggaacaggatagcaggaggctcaggctagcaagtctggactcaacctaaagccagaggcatggttgatagcagagaaagtgaggctcttcacaagtgggtgtgcttaagtaatcagaaacaggaaggctctggttgatggaattatcagtaagatatctacccttatctccttcttctatagaagctaaaccgtctctccttcttgtgtgtaggctgataaacacgcttgttttcttttgagtgttcatggctttgcagattttcagtgctctgccagttcttgttagagggtttgttaccttgacacctgggcttggatgttagcatgccaaaggcacacacttctgaatgcctgtgtaaaaggttattattcatttactttgtctttggaaaggtgaagtgtgtgtgagaaagaactcacaggagatgtattctctgtaggaaaacttttttttccccttaaaagcctataatccactttcagtcaactttgacttttataccatgctgtcacatgaaagagtgtttaggcccgctctcgtggctctgggaaaagcaccaataggggaagaaatgttatgccgagaaatctgactggcagggaaactgggtcagagctccccaaagaccactacaggtgttaagtaggaacagtcgagggtgggttcatataatagaatggaacagagggagggaagataagctacaaagtttcatagggtcctaagtctttaagatacaaaatagctggttgggcttcataacaaaggaagtctgggaaggcagcaagcattgagagggagatggaaagggaaaaaacaatgtagaggatttgaaaagctacaaatcctccacgagaggatttttcttggaggaatctagaacaagggtggtggattaggtggatcgcagaaggacttgctttgccatttgaatctgggtttttgtctctccattgaggttgagagcgtcacccttttttaccctggataggaggaggaaagaaggggtgttttgactcctacctggagttttactagtttacgcaatggaacagacactcgggacctcctcttgacaagaaaaaaaaaaaaaaaaggaaacctgttgtttctcttgtttgttcttttgttaagaaagcacaggcagctgggcatggtggcccatgcctttaatcccagcatttgggaggcagaggcaggtgactttctaaattcaaggccagcctggtctacaaagtgagttccaggacagccagggctatacagagaaaccctgtctcgggaaaaaaaaaaaagaagaaaagaaaagaaaagaagagaagaggagaggagaggagaggagaggagaggagaggagaggagaggagaggagaggagaggagaggagaagagaagagaagagaagagaagagaagagaagagaagagaagagaagagaagagaagagaagagaagagaagagaagagaagagaagagaagagaaaagaaaagagaaaagaaaagaaaaaagcaagcaagcaagcactggcaaagcatgcccacatgggacgtatgtgggtctttgagacaaggcttttgaattgagcgctcatcaatagttgatcatggtcaggtggagggctacctgtcaggccgagccctgctggcttagcacttaacatctccaggtctcagtatcacttcctgctgcttagcacagttaggagttgagcaaacctttttttccaacccccactaaaatttaatttacaaaaggcagtgtaatttgtgggatacagtgtgataattgatctatgtgtgcattgtgcaaggttcaataaggtagatcaataggcccatcaacagctttatgggtgtgaaatgcaagtaatataggtagatgcctgtgtgtccttaggtcagaaaggcatgattttaaggtcttgggcaaatcatattatactcatgttaaaaatgcattatgttgattatcaatcttttagagaaggctgatacttggttttggtgctcagcaagcaaatgtcaccagctctttctaactagtaccactttagaaaatgctacccgtgctcaaattggtttgtattcttattttcatagcttggagagagcggagagatcaaagcgatcggggaactggacctgctgtttatgtctctgagaaatgcttgcgtctgagcgagaagaagctagaaaacgaagaactgctccttcctgccttctaaaaagaacaataagatccctgaatggacttttttactaaaggaaagtgagaagctaacgtccaccatcattagaagatttcacatgaaacctggctcagttgaaagagaaaatagtgtcaagttgtccatgagaccagaggtagacttgataaccacaaagattcattgacaatattttattgtcattgataatgcaacagaaaaagtatgtactttaaaaaattgtttgaaaggaggttacctctcattcctctagaagaaaagcctatgtaacttcatttccataaccaatactttatatatgtaagtttatttattataagtatacattttatttatgtcagtttattaatatggatttatttatagaaaaattatctgatgttgatatttgagtataaagcaaataatatttatgataataactatagaaacaagatatcttaggctttaataaacacatgaatatcataaatcttctgtcttgtaatttttctccctttaatatcaacaataccatcatcgtcatcattacccaatcattctcatgacttcatgcttgactcatattatctggtaaagtttg

SEQ ID NO: 43 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

(19) United States
(12) Patent Application Publication
(10) Pub. No.: US 2005/0271619 A1
(43) Pub. Date: Dec. 8, 2005

(54) Isolated nucleic acid molecules which encode T cell inducible factors, or interleukin-21, the proteins encoded, and uses thereof

(76) Inventors: Laure Dumoutier, Brussels (BE); Jean-Christopher Renauld, Brussels (BE)

Correspondence Address: Norman D. Hanson;FULBRIGHT & JAWORSKI L.L.P., 666 Fifth Avenue, New York, NY, 10103, US

(21) Appl. No.: 11/177,987

(22) Filed: Jul. 7, 2005

Related U.S. Application Data
(63) Continuation of application No. 09/626,617, filed on Jul. 27, 2000.
Application No. 09/626,617 is a continuation-in-part of application No. 09/419,568, filed on Oct. 18, 1999, now Pat. No. 6,331,613.
Application No. 09/419,568 is a continuation-in-part of application No. 09/354,243, filed on Jul. 16, 1999, now Pat. No. 6,359,117.
Application No. 09/354,243 is a continuation-in-part of application No. 09/178,973, filed on Oct. 26, 1998, now Pat. No. 6,274,710.

(51) Int. Cl. A61K 38/20

(52) U.S. Cl. 424/85.2

(57) ABSTRACT

The invention involves isolation of nucleic acid molecules, the expression of which are upregulated by interleukin-9. The amino acid sequences of the proteins which correspond to the nucleic acid molecules show some structural features of cytokines. In addition to the nucleic acid molecules and the proteins, various uses of the molecules are disclosed. The molecules are referred to as T cell inducible factors. The molecules are implicated in activation of STAT molecules, acute phase proteins, and inflammation.

### RELATED APPLICATIONS

This application is a continuation in part of Ser. No. 09/419,568, filed Oct. 18, 1999, which is a continuation in part of Ser. No. 09/354,243, filed on Jul. 16, 1999, which in turn is a continuation in part of Ser. No. 09/178,973, filed Oct. 26, 1998. All of these applications are incorporated by reference in their entirety.

### FIELD OF THE INVENTION

This invention relates to newly isolated nucleic acid molecules and their uses. The nucleic acid molecules are shown to be upregulated by the cytokine interleukin-9 (“IL-9”). Also disclosed are the proteins encoded thereby. These molecules have been described as “T Cell Derived Inducible Factors” or “TIFs”; however, as of now, they are known as “interleukin-21” or “IL-21”, as well as “IL-TIF.” For this reason, the terms will be used interchangeable herein. These nucleic acid molecules encode proteins which induce STAT activation in cells. They can be used, for example, in the stimulation of regeneration of targeted tissues. Further, their inhibitors or antagonists can be used to retard, prevent or inhibit differentiation of other tissues.

### BACKGROUND AND PRIOR ART

The last decade has seen knowledge of the immune system and its regulation expand tremendously. One area of particular interest has been that of research on the proteins and glycoproteins which regulate the immune system. One of the best known families of these molecules are the cytokines. These are molecules which are involved in the “communication” of cells with each other. The individual members of the cytokine family have been found to be involved in a wide variety of pathological conditions, such as cancer and allergies. Whereas sometimes the cytokines are involved in the pathology of the condition, they are also known as being therapeutically useful.

Interleukins are one type of cytokine. The literature on interleukins is vast. An exemplary, but by no means exhaustive listing of the patents in this area includes U.S. Pat. No. 4,778,879 to Mertelsmann et al.; U.S. Pat. No. 4,490,289 to Stern; U.S. Pat. No. 4,518,584 to Mark et al.; and U.S. Pat. No. 4,851,512 to Miyaji et al., all of which involve interleukin-2 or “IL-2.” Additional patents have issued which relate to interleukin-1 (“IL-1”), such as U.S. Pat. No. 4,808,611 to Cosman. The disclosure of all of these patents are incorporated by reference herein. More recent patents on different interleukins include U.S. Pat. No. 5,694,234 (IL-13); U.S. Pat. No. 5,650,492 (IL-12); U.S. Pat. Nos. 5,700,664, 5,371,193 and 5,215,895 (IL-11); U.S. Pat. Nos. 5,728,377, 5,710,251, 5,328,989 (IL-10); U.S. Pat. Nos. 5,580,753, 5,587,302, 5,157,112, 5,208,218 (IL-9); U.S. Pat. Nos. 5,194,375, 4,965,195 (IL-7); U.S. Pat. Nos. 5,723,120, 5,178,856 (IL-6), and U.S. Pat. No. 5,017,691 (IL-4). Even a cursory review of this patent literature shows the diversity of the properties of the members of the interleukin family. One can assume that the larger cytokine family shows even more diversity. See, e.g., Aggarwal et al., ed., Human Cytokines: Handbook For Basic And Clinical Research (Blackwell Scientific Publications, 1992), Paul, ed., Fundamental Immunology (Raven Press, 1993), pg 763-836, “T-Cell Derived Cytokines And Their Receptors”, and “Proinflammatory Cytokines and Immunity.” All cited references are incorporated by reference.

The relationships between various cytokines are complex. As will be seen from the references cited herein, as the level of a particular cytokine increases or decreases, this can affect the levels of other molecules produced by a subject, either directly or indirectly. Among the affected molecules are other cytokines.

The lymphokine IL-9, previously referred to as “P40,” is a T cell derived molecule which was originally identified as a factor which sustained permanent antigen independent growth of T4 cell lines. See, e.g., Uyttenhove et al., Proc. Natl. Acad. Sci. 85: 6934 (1988), and Van Snick et al., J. Exp. Med. 169: 363 (1989), the disclosures of which are incorporated by reference, as is that of Simpson et al., Eur. J. Biochem. 183: 715 (1989).

The activity of IL-9 was at first observed on restricted T4 cell lines, failing to show activity on CTLs or freshly isolated T cells. See, e.g., Uyttenhove et al., supra, and Schmitt et al., Eur. J. Immunol. 19: 2167 (1989). This range of activity was expanded when experiments showed that IL-9 and the molecule referred to as T cell growth Factor III (“TCGF III”) are identical to MEA (Mast Cell Growth Enhancing Activity), a factor which potentiates the proliferative response of bone marrow derived mast cells to IL-3, as is described by Hültner et al., Eur. J. Immunol. and in U.S. Pat. No. 5,164,317, the disclosures of both being incorporated by reference herein. It was also found that the human form of IL-9 stimulates proliferation of megakaryoblastic leukemia. See Yang et al., Blood 74: 1880 (1989). Work on IL-9 has shown that it also supports erythroid colony formation (Donahue et al., Blood 75(12): 2271-2275 (6-15-90)); promotes the proliferation of myeloid erythroid burst formation (Williams et al., Blood 76: 306-311 (9-1-90); and supports clonal maturation of BFU-E's of adult and fetal origin (Holbrook et al., Blood 77(10): 2129-2134 (5-15-91)). Expression of IL-9 has also been implicated in Hodgkins's disease and large cell anaplastic lymphoma (Merz et al., Blood 78(8): 1311-1317 (9-1-90). Genetic analyses of mice that were susceptible or resistant to the development of bronchial hyperresponsiveness have unraveled a linkage with the IL-9 gene as well as a correlation between IL-9 production and susceptibility in this model (Nicolaides et al., Proc. Natl. Acad. Sci. USA, 94, 13175-13180, 1997). Human genetic studies also point to the IL-9 and IL-9R genes as candidates for asthma (Doull et al., Am. J. Respir. Crit. Care Med., 153, 1280-1284, 1996; Holrovd et al., Genomics 52, 233-235, 1998). Also, IL-9 transgenic mice allowed for the demonstration that increased IL-9 expression result in lung mastocytosis, hypereosinophilia, bronchial hyperresponsiveness and high levels of IgE (Temann et al., J. Exp. Med. 188, 1307-1320, 1998; Godfraind et al., J. Immunol. 160, 3989-3996, 1998; McLane et al., Am. J. Resp. Cell. Mol. 19:713-720 (1999). Taken together, these observations strongly suggest that IL-9 plays a major role in this disease Additional work has implicated IL-9 and muteins of this cytokine in asthma and allergies. See, e.g. PCT Application US96/12757 (Levitt, et al), and PCT US97/21992 (Levitt, et al), both of which are incorporated by reference.

IL-9 is known to affect the levels of other molecules in subjects. See Louahed et al., J. Immunol. 154: 5061-5070 (1995; Demoulin et al., Mol. Cell. Biol. 16: 4710-4716 (1996), both incorporated by reference. It will be recognized that the molecules affected have their own functions in biological systems. For example, Demoulin et al. show that many of the known activities of IL-9 are mediated by activation of STAT transcription factors. As such, there is continued interest in trying to identify molecules whose presence and/or level is affected by other molecules, such as cytokines.

The disclosure which follows describes such molecules. It was found that nucleic acid molecules encoding the proteins of the invention were expressed in the presence of IL-9, but not in its absence. Hence, these molecules are, inter alia, “markers” for the expression or effect of IL-9 in a subject. The molecules once referred to as T Cell Derived Inducible Factors or “TIFs” have now been referred to as interleukin-21, or “IL-21.” These and other features of the invention will be seen in the disclosure which follows.

### DETAILED DESCRIPTION OF PREFERRED EMBODIMENTS

### EXAMPLE 1

The murine lymphoma cell line BW5147 is well known as a cell line which can be grown in vitro, without the need to add any cytokines to its culture medium. In order to identify genes induced by IL-9, samples of BW5147 were cultured either with (200 U/mil), or without IL-9, for 24 hours. Then, total RNA was isolated, using guanidium isothiocyanate lysis, and CsCl gradient centrifugation. These techniques are well known in the art. Following this, polyadenylated RNA was purified from the total RNA, by using an oligo(dT) cellulose column. The isolated, polyA RNA was then used to generate double stranded cDNA. A commercially available oligo(dT) primer was used. Anywhere from 3-5 μg of polyA RNA were heated to 70° C. for 10 minutes with 1 μg of oligo (dT), and then incubated with 5× first strand buffer (250 mM HCl (pH 8.3), 375 mM KCl, 15 mM MgCl₂), 10 mM dithiothreitol, 500 μM of deoxynucleotide triphosphates, and 800 U of reverse transcriptase. Total volume of the reaction mixture was 20 μl, and the reaction was allowed to proceed at 37° C. for one hour. This resulted in synthesis of the first strand of cDNA. Second strand synthesis was accomplished by adding 30 μl of 5 second strand buffer (100 mM Tris-HCl (pH 6.9)), 450 mM KCl, 23 mM MgCl₂, 0.75 mM β-NAD⁺, 50 mM (NH₄)₂SO₄, together with 60 U of E. coli derived DNA polymerase I, 2 U of E. coli RNase H, 10 U of E. coli DNA ligase, and 250 μM of deoxynucleotide triphosphates, and brought to a final volume of 150 μl. The mixture was incubated for two hours, at 16° C.

The product was extracted using phenol-chloroform, and was precipitated with ethanol. The final cDNA product was then resuspended in 200 μl of TE.

These steps were carried out for both the stimulated BW5147 cells (“tester” hereafter), and for parallel, unstimulated BW5147 cells (“driver” hereafter).

### EXAMPLE 2

The cDNA prepared in Example 1 was then subjected to subtraction cloning in accordance with well known methods. To do this, six oligonucleotides were prepared:

| | |
|---|---|
| 5′-AGCACTCTCC AGCCTCTCAC CGCA-3; | (SEQ ID NO: 1) |
| 5′-GATCTGCGGT GA-3′; | (SEQ ID NO: 2) |
| 5′-ACCGACGTCG ACTATCCATG AACA-3′; | (SEQ ID NO: 3) |
| 5′-GATCTGTTCA TG-3′; | (SEQ ID NO: 4) |
| 5′-AGGCAACTGT GCTATCCGAG GGAA-3′; | (SEQ ID NO: 5) |
| and | |
| 5′-GATCTTCCCT CG-3′. | (SEQ ID NO: 6) |

These were used as explained herein. Double stranded cDNA (2 μg), was digested with restriction endonuclease DpnII, extracted with phenol-chloroform, precipitated with ethanol, and resuspended in 20 μl of TE (10 mM Tris-HCl (pH 7.5); 1 mM EDTA). Twelve μl (1.2 μg), of cut cDNA was ligated to double stranded SEQ ID NOS: 1 and 2, in a mixture which included 4 μl of desalted SEQ ID NO: 1 (2 mg/ml), 4 μl desalted SEQ ID NO: 2 (1 mg/ml), 10 μl of 5× adapter buffer (330 mM Tris-HCl, pH 7.6, 50 mM MgCl₂, 5 mM ATP), 7 μl DTT (100 mM), and 28 μl of H₂O). The oligonucleotides were annealed to each other and to the sample DNA by heating the mixture to 50° C. and then cooling it to 10° C. over one hour, followed by adding 5 μl of T4 DNA ligase, and incubation for 12-14 hours, at 12-16° C. The mixtures were diluted by adding 140 μl of TE. PCR was then carried out on 200 μl samples, as described infra.

### EXAMPLE 3

To carry out PCR, 200 μl samples containing 2 μl of the ligation product in a buffer of 66 mM Tris-HCl, pH 8.8, 4 mM MgCl₂, 16 mM (NH₄)₂SO₄, 33 μg/ml BSA, 0.3 mM of each dNTP (concentration: 500 μM), and 2 μg of SEQ ID NO: 1 were first heated at 72° C. for three minutes to remove any of SEQ ID NO: 2 which was hybridized to the product of Example 2. The 3′ ends were then filled in by using 5 U of Taq polymerase (5 minutes, 72° C.). Twenty cycles of amplification were carried out (1 cycle:1 minute at 95° C., and three minutes at 72° C.), after which products were combined, phenol extracted, ethanol precipitated, and resuspended in TE buffer, at a concentration of 0.5 μl/μl. Hereinafter, this is referred to as the representation.

### EXAMPLE 4

The representation was then prepared for subtractive hybridization by removing SEQ ID NO: 1 therefrom by digestion with Dpn II. The resulting digest was phenol extracted and ethanol precipitated. In the case of the unstimulated sample, this resulted in the driver, while the stimulated sample resulted in the tester. Portions of tester (20 μg) were gel purified on a 1.2% agarose gel and isolated. Samples (2 μg), were ligated to SEQ ID NOS: 3 and 4, in the same way that SEQ ID NOS: 1 and 2 were ligated, as described, supra.

In a first cycle of subtractive hybridization, 0.4 μg samples of tester with SEQ ID NOS: 3 and 4 ligated thereto were mixed with 40 μg of driver cDNA. The mixture was phenol extracted, ethanol precipitated, dissolved in 2 μl of 3×EE buffer (30 mM EPPS pH 8.0), 3 mM EDTA; pH 8.0, 3 mM EDTA. This was overlaid with 30 μl of mineral oil, and denatured for five minutes at 98° C. A 5M NaCl solution (0.5 μl) was added, and DNA was hybridized for 20 hours, at 67° C. The reaction mixture was diluted to 200 μl with TE, and tRNA carrier. The samples were incubated for three minutes at 72° C. to melt away SEQ ID NO: 4, and then four PCR reactions (200 μl) were prepared. These included 20 μl of diluted hybridization mix without primer, to fill in the ends of the reannealed tester, followed by 10 cycles of amplification after adding samples of SEQ ID NO: 3 (1 cycle:1 minute at 95° C., three minutes at 70° C.) after which products were combined, phenol extracted, ethanol precipitated, and resuspended in 40 μl of 0.2×TE buffer. Single stranded DNA was degraded by a 30 minute treatment of 20 μl of this material with 20 U of mung bean nuclease, at a total volume of 40 μl. Samples was diluted (1:5), in 50 mM Tris-HCl, at pH 8.9, followed by five minutes of heating at 98° C. to inactivate the enzyme. A second PCR was carried out, using 20 μl of the product described supra, 2 μl of SEQ ID NO: 3 (1 mg/ml), and 1 μl (5 U) of Taq DNA polymerase. A total of 18 cycles (1 cycle: 1 minute at 95° C., three minutes at 70° C.) were carried out. Products were combined, phenol extracted, ethanol precipitated, and resuspended at 0.5-1 μg/μl. The product is referred to hereafter as “DP1”, or the first difference product.

### EXAMPLE 5

DP1 was then digested with endonuclease DpnII, as described above, and was ligated to SEQ ID NOS: 5 and 6, following the same processes described for SEQ ID NOS: 1, 2, 3 and 4. Subtractive hybridization and selective amplification, as described in example 4, was repeated, and second difference product, or “DP2”, was generated. In these experiments, 50 ng of DP1 was the tester. The driver (40 μg), was as described supra. The process was repeated to generate a third difference product, using SEQ ID NOS: 3 and 4 as adapters. To generate the third product, 100 pg of tester were mixed with 40 μg of driver. All steps of the protocols supra were repeated, except the final amplification was carried out for 22 cycles, where one cycle was one minute at 95° C., and three minutes at 70° C. This yielded the final difference product.

### EXAMPLE 6

The final difference products were digested with DpnII, and then cloned into the BamHI site of a commercially available vector, i.e., pTZ19R. Double stranded DNA plasmids were prepared, and then sequenced, using standard methods. The sequences were compared to known sequences in the GenBank and EMBL data bases, using a BLAST search program.

At the end of this subtraction procedure, a short cDNA fragment was identified, i.e., a fragment about 200 base pairs long. This fragment was used to screen a cDNA library from BW 5147 cells. The largest clone was sequenced. It is discussed infra. It does not correspond to any known sequence.

The nucelotide sequence (SEQ ID NO: 7), is 1119 bases long, including a 537 base pair open reading frame, which encodes a protein 179 amino acids long. The predicted molecular weight of the protein is 20,093. There are two additional ATG codons which, if they acted as start codons, would produce proteins 172 and 167 amino acids in length, with molecular weights of 19,335 and 18,770 daltons, respectively. Each form of the protein is characterized by a sequence of hydrophobic amino acids which would be cleaved off of the molecule via the endoplasmic reticulum to provide a mature protein.

Analysis of the sequence shows three AT rich motifs (TTATTTAT). These motifs are often found in 5′-untranslated regions of cytokines and oncogenes. Kruys, et al., Science 245: 852 (1989), have shown that these repeats modulate stability of mRNA.

### EXAMPLE 7

The cDNA isolated and analyzed in example 6, supra, was then used as a probe to identify genomic DNA for TIFα.

A genomic library prepared from mouse strain 129 was screened with SEQ ID NO: 7, following standard methods. An EcoRI fragment from a positive clone was subcloned into plasmid pZERO and partially sequenced. The partial sequence is presented as SEQ ID NO: 8.

### EXAMPLE 8

A second EcoRI fragment from the positive clone described in Example 7, supra, was also subcloned. There was a great deal of homology, but the sequences were not identical. To be specific, intron 1 of this sequence was 98% identical to SEQ ID NO: 8, intron 2 was 100% identical and intron 3 was 92% identical.

What is striking about the sequences is that the promoters are not at all homologous, suggesting independent regulation. The 5′ untranslated regions are 92% identical. The first exon for TIFα is split into exon 1α and exon 1β. The first coding exon (which is exon 1b for TIFα and exon 1 for TIFβ) are 99.5% identical, while the second exons are 100% identical, the third exons 97% identical, the fourth exons 98.5% identical, and 96% for the fifth exon. In the untranslated 3′-region, homology is 96%.

### EXAMPLE 9

Using the information described in example 8, supra, a cDNA sequence for the second clone, designated TIFβ was deduced, and is set forth as SEQ ID NO: 9. The genomic DNA sequence was also ascertained, in the same manner as is described, supra, and is set forth as SEQ ID NO: 42. The amino acid sequence is SEQ ID NO: 41.

As compared to the coding region for TIFα, that of TIFβ has six silent changes. There are two changes which result in an inconsequential amino acid change (at both of positions 36 and 103, Val in TIFα becomes Ile in TIFβ). There is also a more significant change, at position 112, where Gln becomes Arg.

### EXAMPLE 10

Experiments were undertaken to study expression of the TIFs. BW 5147 cells were stimulated with recombinant murine IL-9 (200 U/ml), for varying periods of time (0.2, 0.5, 1, 2 & 24 hours). Total RNA was then isolated, using standard methods and reagents. Reverse transcription was then carried out, using 51 μg total RNA and an oligo (dT) primer. Samples of cDNA corresponding to 20 ng of total RNA were then amplified for 25 cycles using different primers. (One cycle was 4 minutes at 94° C., 1 minute at 57° C., and 2 minutes at 72° C.). The TIF primers were:

| | |
|---|---|
| 5′-CTGCCTGCTT CTCATTGCCC T-3′ | (SEQ ID NO: 10) |
| and | |
| 5-CAAGTCTACC TCTGGTCTCA T-3′ | (SEQ ID NO: 11) |

(sense and antisense, respectively).

These correspond to nucleotides 107-127, and 766-786 of SEQ ID NO: 7, respectively. As a control, β-actin was amplified as well, for 18 cycles (first cycle: 4 minutes at 94° C., 1 minute at 60° C., 2 minutes at 72° C. Succeeding cycles were 1 minute at 94° C., 1 minute at 60° C., 2 minutes at 72° C.).

Following amplification, post PCR products were analyzed on a 1% agarose gel, and specific amplification was confirmed, following blotting, using internal radioactive probes. The probe for TIF was:

| | |
|---|---|
| 5′-GACGCAAGCA TTTCTCAGAG-3′ | (SEQ ID NO: 12) |

the conditions and probes set forth were not specific for one or the other of the forms of TIF; however, the amplification product of TIFα contains a KpnI restriction site, while the restriction site for TIFβ does not. Digestion of the amplification products with KpnI indicated that most, if not all, of the TIF mRNA induced by IL-9 was TIFα, suggesting that the TIFα expression was induced rapidly via the IL-9. The mRNA for TIFα was detectable after 30 minutes of stimulation, and reached a plateau over a 1-24 hour time period.

### EXAMPLE 11

Experiments were then carried out which showed that the induction of TIF mRNA by IL-9, described supra, does not require protein synthesis. In these experiments, total RNA was extracted from cells stimulated for 24 hours, as described in example 10, but with or without 10 μg/ml of a protein synthesis inhibitor, cycloheximide, for 4.5 hours. In a parallel set of experiments, cells were not stimulated. The total RNA was extracted, and RT-PCR amplification was carried out as described in example 10. Post-PCR products were analyzed on an ethidium bromide-stained, 1% agarose gel. What was seen was that the induction by IL-9 still occurred when protein synthesis was blocked. Hence, the effect of IL-9 is a direct effect, not requiring the synthesis of a protein mediator.

### EXAMPLE 12

In these experiments, the role of STAT proteins in induction of TIF mRNA was studied on derivatives of the cell line BW5147. The first line, BWh9R, expresses wild type human IL-9 receptors. The line BW-Phe 116 is a transfectant with a single mutation (at position 116), which renders the receptor unable to activate STAT transcription factors. Still another cell line, BW-mut6, has a mutation which renders the receptor unable to activate STAT5, while retaining the ability to activate STAT1 and STAT3. Finally, cell line BW-mut7 has a single mutation which renders the IL-9 receptor unable to activate STAT1 and STAT3, but which retains the ability to activate STAT5.

Cell stimulation, isolation of total RNA, reverse transcription and amplification of cDNA were all carried out as described in example 10 (Cells were stimulated for 24 hours. Both human and murine recombinant IL-9 were used). The PCR products were analyzed on an ethidium bromide stained, 1% agarose gel, as describe supra.

The analysis revealed that human IL-9 did not induce expression in BW-Phe116, suggesting that STAT transcription factors are implicated. It was found that IL-9 induced TIF expression in the BW-mut6 mutant, but not the mut7 variant, suggesting that STAT1 or STAT3 are involved, but not STAT5.

### EXAMPLE 13

The expression of TIF mRNA in normal mouse spleen cells was then studied.

Spleen cells from 10-12 week old Balb/c mice were cultured for 24 hours in control medium or the control medium supplemented with 20 g/ml of LPS (which activates B lymphocytes and macrophages), or ConA (which activates T cells), or ConA plus 1% of a blocking antiserum against murine IL-9, with β actin being used as a control. Purification of RNA, RT-PCR analysis were carried out as described supra.

The data indicated that TIF is, at best, very weakly expressed in resting spleen cells, not induced by LPS, but strongly induced by ConA. Anti IL-9 antiserum did not affect induction by ConA, suggesting that its effect is not mediated by IL-9, or is mediated by other cytokines.

When the ConA activated spleen cells were analyzed using sequences of RT-PCR products, it was found that these cells were expressing TIFα predominantly, or exclusively.

### EXAMPLE 14

Further experiments showed that TIF mRNA was expressed even in the absence of IL-9 induction.

Spleen cells from 5 week old FVB mice were enriched for T cells, using a nylon wool column. Then, the cells were stimulated for 24 hours in medium supplemented with ConA (a T cell activator), or PMA (which activates PKC in most cells), either with or without IL-9.

Total RNA was isolated using standard techniques, and then ten microgram samples were fractionated via electrophoresis on a 1.3% agarose gel containing 2.2M formaldehyde. The fractions were then transferred to a nitrocellulose membrane, labeled, and assayed in a hybridization assay following Van Snick, et al, J. Exp. Med. 169: 363 (1989), incorporated by reference.

The results indicated that the induction of TIF by ConA was not modified, and that IL-9 did not induce TIF RNA in PMA activated spleen cells.

### EXAMPLE 15

The expression of TIF mRNA in various cell lines was tested. In these experiments, murine cell lines were stimulated for at least one day, with a particular cytokine. Specifically, 9T7 is a T cell lymphoma, which responds to IL-2, IL-4 or IL-9. Cell lines TS3 and TS6 are derived from T helper cell clones, and proliferate in the presence of either IL-2 or IL-9. MC9 and L138 are mast cell lines, which proliferate in the presence of either IL-3 or IL-9.

Following stimulation, total RNA was prepared using standard guanidium isothiocyanate lyses, and CsCl gradient centrifugation.

The 9T7 line was then analyzed by Northern blotting, as described in example 14, while the other lines were assayed using RT-PCR analysis, as described supra.

It was found that IL-9 upregulated TIF expression in T helper cells and mast cells, while IL-2 and IL-3 did not. The 9T7 cell line, however, showed roughly the same level of expression, regardless of the cytokine, indicating that IL-9 is not mandatory for TIF expression.

### EXAMPLE 16

The expression of TIF mRNA in B cell lines was then studied. The cell lines A20, 70Z/3, and BCL-1 are B cell leukemia cell lines which grow, in vitro, without cytokines. These cells were stimulated for 24 hours with IL-4 and IL-9 and total RNA was isolated, using standard methods. Expression was analyzed by RT-PCR which was carried out for 35 cycles, followed by blotting and hybridization, as described supra.

The results indicated that TIF expression is detectable in B cells, but is weakly upregulated at best in the presence of IL-9 and IL-4.

### EXAMPLE 17

Experiments were then carried out to study expression of the inventive molecules in T helper cell lines. TS2 and TS1 are known T helper cell lines, derived from T helper cell clones, which proliferate in the presence of either IL-9 or IL-2 (TS2), and either IL-9 or IL-4 (TS1). Specifically, TS1 or TS2 cells were grown in the presence of the listed cytokines for at least 10 days, after which RNA was extracted using known methods. Expression of the molecules was studied via RT-PCR (35 cycles), using the protocols described supra. In TS1 cells both IL-4 and IL-9 induce TIF expression, but IL-2 does not do so in TS2 cells.

### EXAMPLE 18

Expression of TIF mRNA in various mouse organs were studied. Total RNA was prepared from liver, kidney, heart, brain, intestine, spleen, thymus, lung, muscle and bone marrow, using standard guanidium isothiocyanate methodologies and CsCl gradient centrifugation. Forty cycles of RT-PCR were carried out, using the protocols described supra. Strongest expression was found in thymus tissue, while less intense signals were found in brain tissue, and weaker expression in the remaining tissues.

### EXAMPLE 19

The following experiments describe production of TIFα in 293-EBNA cells.

Complementary DNA for TIFα was described supra. It was subcloned into a commercially available expression vector pCEP-4, in operable linkage with a CMV promoter. The resulting plasmids were transfected into 293-EBNA cells, using standard lipofectamine methods. Following transfection, the cells were incubated in a methionine free medium, supplemented with ³⁵S labeled methionine, for 24 hours. Supernatant was harvested, and run on an acrylamide gel, followed by electrophoresis. The gel was then dried and exposed to autoradiography for 1 day. A control was then run by transfecting cells with the same plasmid, in which the cDNA was cloned in the antisense direction.

A heterogenous band of about 25-30 kilodaltons was found from the cells transfected with TIF in the sense direction. Any discrepancies between the predicted molecular weight, the actual molecular weight in the system, and the heterogeneity, can be attributed to glycosylation. In a series of parallel experiments, cDNA encoding human TIF was expressed in the same way as the murine cDNA was expressed. With the exception of the change of the cDNA, all experimental parameters were the same.

### EXAMPLE 20

Further experiments were carried out to study production of TIFα in COS cells. Specifically, TIFα cDNA was subcloned into the plasmid pEF-BOS.puro described by Demoulin et al., supra, in operable linkage with the EF-1α promoter. The plasmid cDNA was transfected into COS cells, using the same lipofectamine method described supra. The cells were incubated in methionine free medium, supplemented with ³⁵S methionine for 24 hours, after which supernatant was treated as described in example 19, supra. Again, a heterogenous band of 25-30 kilodaltons was observed, as well as an 18 kilodalton band, which probably represents a non-glycosylated form of the molecule.

### EXAMPLE 21

In these experiments, it was discovered that TIF induces STAT activation in mesangial, neuronal melanoma, and hepatoma cells. It is known that when cytokines activate STAT factors, the factors dimerize, move from cytoplasm to the nucleus, and bind to target sequences in promoters. The details of the experiments follow.

Transfected 293-EBNA cells as described supra were used following incubation in normal medium for 48 hours, as were supernatant from the controls, also described supra. Samples of a mouse kidney mesangial cell line, (“MES13” hereafter), a rat pheochromocytoma cell line, (“PC12” hereafter), four different human melanomas (SK23, AUMA, NA-8mel and MULL), human hepatoma (HepG3) and rat hepatoma (H-4-II-K) were used. Cell samples (0.5×10⁶) were stimulated for 5-10 minutes in the presence of 1% of supernatant. Nuclear extracts were then prepared, in accordance with Demoulin et al., Mol. Cell. Biol. 16: 4710 (1996), incorporated by reference. In brief, cells were washed with PBS and then resuspended in 1 ml of ice cold hypotonic buffer for 15 minutes. (Buffer was 10 mM HEPES buffer, pH 7.5, with 10 mM KCl, 1 mM MgCl₂, 5% glycerol, 0.5 mM EDTA, 0.1 mM EGTA, 0.5 mM dithiothreitol, and 1 mM Pefabloc, 1 mM Na₃V₄, and 5 mM NaF). Cells were then lysed by adding 65 μl of NP-40, followed by vortexing. Nuclei were pelleted, by vortexing for 30 seconds at 14,000 rpm, followed by extraction in buffer supplemented with HEPES (20 mM), glycerol (20%), and NaCl (420 mM). Nuclear debris was removed by centrifuging for 2 minutes. DNA binding activity was determined in accordance with Demoulin et al., supra, using a ³²P labeled double stranded oligonucleotide called “GRR,” which contains the STAT binding site of the FcγRI gene promoter, i.e.:

| | |
|---|---|
| 5′ATGTATTTCC CAGAAA-3′ | (SEQ ID NO: 13) |
| and | |
| 5′-CCTTTTCTGG GAAATAC-3′ | (SEQ ID NO: 14) |

corresponding to the upper and lower strands of the binding sites in the GRR probe. Briefly, 5 μl volume of nuclear extracts were incubated in binding buffer (12 mM HEPES, pH 7.6, 10 mM KCl, 0.5 mM EDTA, 2.5% glycerol, 0.1 mg of poly(dI-dC) per ml) for 5 minutes. Radiolabeled GRR probe (10⁵ cpm; approximately 0.5 ng) was added, and incubation was continued for 25 minutes before loading onto a non-denaturing polyacrylamide gel.

It was also noted that the complexes observed in MES13 cells, described supra, were partially overshifted by both anti-STAT5 and anti-STAT3 antibodies, showing that (i) the cells under examination were targets for TIF, and (ii) that STAT3 and STAT5 are major components of the complex activated by TIF. The difference in STAT profile, as compared to the profile in Example 12, supra, is attributable to the difference in cell source (human versus mouse). It was also observed that human TIF works on murine cells, and vice versa.

### EXAMPLE 22

This example details the isolation and cloning of a nucleic acid molecule which encodes human TIF. First, human peripheral blood mononuclear cells were prepared via standard density gradient centrifugation. Following this preparation, samples were cultured for 24 hours, at 3×10⁶ cells/ml, either with or without anti-CD3 monoclonal antibody (The antibody was the commercially available OKT3 mAb, used in the form of ascites fluid at 1/500 dilution). This antibody was used because T cell derived cytokines are generally expressed only upon activation by e.g., CD3 specific antibodies.

Total RNA was isolated from these cells, using standard guanidine-isothiocyanate/CsCl ultra-centrifugation techniques. Following isolation, 10 μg samples of the RNA were reverse transcribed using an oligo (dT)15 primer.

Following preparation of cDNA, as outlined supra, samples which corresponded to 100 ng of total RNA were amplified, via PCR, using the following primers:

| | |
|---|---|
| 5′ - AGGTGCTGAA CTTCACCCTG GA - 3′ | (SEQ ID NO: 15) |
| 5′ - CCACTCTCTC CAAGCTTTTT CA - 3′ | (SEQ ID NO: 16) |

which are based upon a murine cDNA sequence, (i.e., SEQ ID NO: 7). The PCR conditions involved 30 cycles of amplification, with one cycle defined as 1 minute at 94° C., followed by 1 minute at 42° C., and then 2 minutes at 72° C. Amplification product was separated on an agarose gel, using standard methods, and then sequenced. The result indicated that fragments of the cDNA had been amplified. Hence, a second reaction was carried but, using the same materials except SEQ ID NO: 16 was replaced by SEQ ID NO: 17, i.e.:

5′ -CAAGTCTACC TCTGGTCTCA T- 3′

This second PCR reaction was carried out for 25 cycles, with one cycle being defined as 1 minute at 94° C., followed by 1 minute at 45° C., and then 2 minutes at 72° C. The amplification product was subjected to the same steps as the first one. A fragment of 418 nucleotides was amplified, which was found in anti-CD3 stimulated cells, but not resting PBMCs. This fragment's nucleotides sequences is set forth at SEQ. ID NO: 18.

### EXAMPLE 23

Following preparation of amplification product, the 5′ end of cDNA was isolated by using standard, 5′-RACE techniques. In brief, first strand cDNA was prepared by using SEQ ID NO: 19 as a primer, i.e.:

5′ - TGGCCAGGAA GGGCACCACC T -3′

This primer was based upon the sequence information obtained in accordance with example 22. In brief, the 5′-RACE method was carried out by combining 1 μg of total RNA, prepared as described supra, 2.5 pmoles of SEQ ID NO: 19, reverse transcriptase, reverse transcriptase buffer, 2.5 μl of dNTP mix (10 mM), 2.5 μl of MgCl₂ (25 mM), and 2.5 μl of dithiothreitol (0.1 M). The reaction was carried out and, after completion, original RNA was removed via adding RnaseH, and Rnase TI. Any unincorporated dNTPs, as well as primer and proteins, were removed. The cDNA was tailed using terminal transferase, or “TdT.” This enzyme creates a 3′-binding site for the abridged anchor primer, as described infra. Tailing was carried out by combining the purified, first strand cDNA, TdT, buffer (10 mM Tris-HCl, 25 mM KCl, 1.5 mM MgCl₂), and 200 μM of dCTP.

Following the tailing reaction, PCR was carried out using:

| | |
|---|---|
| 5′ -CCTATCAGAT TGAGGGAACA G -3′ | (SEQ ID NO: 20) |

and 5′-RACE abridged anchor primer:

(SEQ ID NO: 21)

5′ - GGCCACGCGT CGACTAGTAC GGGIIGGGIIGGGIIG -3′.

The amplification involved 35 cycles (1 cycle defined as 1 minute at 94° C., 1 minute at 56° C., and 2 minutes at 72° C.). Following this, nested amplification was performed on 5 μl of a 1/100 dilution of the amplification product, using SEQ ID NO: 20 and the abridged universal amplification primer:

| | |
|---|---|
| 5′ - GGCCACGCGT CGACTAGTAC -3′. | (SEQ ID NO: 22) |

Amplification involved 30 cycles (1 cycle being defined as 1 minute at 94° C., 1 minute at 56° C., and 2 minutes at 72° C.). The resulting PCR product was cloned, following standard procedures, and sequenced.

These three protocols, i.e., the two experiments described supra which generated fragments, and the 5′-RACE PCR, also described supra, permitted alignment of the sequenced amplification product, to generate the complete sequence.

Following the alignment, oligonucleotides were generated which flanked the deduced open reading frame, i.e.:

| | |
|---|---|
| 5′ - CCTTCCCCAG TCACCAGTTG -3′ | (SEQ ID NO: 23) |
| and | |
| 5′ - TAATTGTTAT TCTTAGCAGG -3′. | (SEQ ID NO: 24) |

These primers were used to amplify the entire open reading frame, using mRNA from CD3 specific mAb stimulated cells, as described supra. For amplification, 25 cycles (1 cycle being defined as 1 minute at 94° C., 1 minute at 56° C., and 2 minutes at 72° C.).

The complete sequence of the human cDNA is set forth at SEQ ID NO: 25. It contains a 537 base pair ORF which encodes a 179 amino acid protein. This is the same length as the murine protein. The human protein has 79% amino acid homology with the murine protein, and 25% homology with IL-10. Human and murine proteins are set out as SEQ ID NOS:43 and 40, respectively.

### EXAMPLE 24

These experiments detail work on the isolation of human genomic DNA corresponding to the cDNA discussed supra.

Based upon the cDNA sequences, primers were developed which correspond to nucleotides 51-70 and the complement of nucleotides 631-650 of SEQ ID NO: 25. PCR was carried out, using standard methodologies. Specifically, 100 ng of genomic DNA taken from CESS cells (an EBV-transformed, lymphoblastoid cell line) was used as a template, and 33 cycles of amplification were carried out (one cycle of amplification being defined as 94° C. for 30 seconds, 50° C. for 30 seconds, and 72° C. for 5 minutes). Once a sequence was isolated, it was sequenced, and this is set forth as SEQ ID NO: 26. The sequence is about 4.8 kilobases in length, and is believed to contain the entire genomic sequence encoding the TIF molecule, lacking only the 5′ flanking region, the promoter, and the 3′ end. Analysis indicates that it contains 6 exons and 5 introns, as does the murine genomic sequence.

Southern blot hybridization was carried out, using standard methods. It showed that the genome contains only a single copy of the TIF gene.

### EXAMPLE 25

It was of interest to identify where the genomic DNA discussed supra was located in the human genome. In order to do this, two different approaches were taken. In the first, the sequence discussed supra, i.e., SEQ ID NO: 26, was labeled with a flourescent label, and then was used to probe the human genome via fluorescent, in situ hybridization (“FISH”) using standard methods.

In a second approach, a panel of radioactive hybrid clones were screened using the probe consisting of nucleotides 51-70 of SEQ ID NO: 25, and 5′-ATCAGATGGA TTACTGAATG-3′ (SEQ ID NO:27). PCR was carried out using 25 ng of genomic DNA as a template, for 35 cycles, where one cycle is defined as 94° C. for 1 minute, 55° C. for 1 minute and 72° C. for 2 minutes.

Both methodologies indicated that the gene is located at chromosome 12q15. Some work links diseases associated with asthma at this site. See, e.g. Nat. Genet. 15:389-392 (1997); Ober, et al, Hum. Mol Genet. 7(9):1393-1398(1998); Nickel, et al, Genomic 46(1):159-162(1997); Takahashi, et al, Genomics 44(1):150-2(1997); Barnes, et al, Genomics 37(1):41-50(1996), all incorporated by reference.

Public databases were consulted to determine if any part of the sequences were presented therein. The last exon of TIF was found on a BAC clone, derived from chromosome 12q15. (Accession No: AC007458; 191,111 base pairs, BAC RDCI11-444B24). The identification of the last exon on this clone suggests that the TIF gene is located about 90 kilobases from the IFN gene, and less than 30 kilobases from a gene referred to as AK155, which is an IL-10 related cytokine (Knappe, et al, J. Virol 74:3881-3887 (2000)).

### EXAMPLE 26

These experiments describe the manufacture of antibodies which bind to the TIF protein. To make these, a peptide consisting of amino acids 40-61 encoded by SEQ ID NO: 7 was coupled to KLH carrier protein, using standard methods and a ratio of 1 mg peptide to 1 mg carrier protein. Subject animals (rabbits), were immunized 3 times, at 2 week intervals, with 150 μg of the complex. The immunogen was emulsified in Complete Freund's Adjuvant for the first injection, and then Incomplete Freund's Adjuvant for the next two.

A first bleed was performed one month after the last injection, and serum was prepared, following known methods.

The serum was then tested in a standard Western Blot. In brief, 10 μl of supernatant from cells transfected with either SEQ ID NO: 7 or SEQ ID NO:25 were separated via SDS-PAGE electrophoresis, and then blotted onto PVDF membranes. Antiserum was diluted to 1:500, and used in a standard Western Blot protocol, together with anti-rabbit antibody as the secondary antibody, and a commercially available detection kit.

It was found that the serum did, in fact, recognize the TIF protein.

### EXAMPLE 27

This example describes experiments carried out to identify cell lines which are responsive to human TIF. HEK293-EBNA human embryonic kidney cells, described supra, were seeded in 6 well plates, at 3×10⁵ cells/well one day before transfection. They were transfected with 2 μg of pCEP-4 plasmid which contained cDNA for human TIF, under control of the CMV promoter. Cells were incubated after transfection, in 1.5 ml normal medium, for 3 days, in order to maximize production of recombinant human TIF.

It was hypothesized that human TIF would induce activation of STAT transcription factors in the same way the murine factor did. The protocol of example 21, supra was followed. Supershifts were performed by adding anti-STAT antibodies (0.75 μg anti STAT 1, 1 μg of anti-STAT 3, or 1 μg of anti-STAT 5b), to mixtures of nuclear extracts and labeled DNA probe, and incubated.

A TIF induced bandshift was observed when the hepatoma cell line HepG2 was used. The antibodies described supra were used to characterize the response further. Anti-STAT-3 antibodies shifted most of the retardation complexes, while the weak, remaining complexes were supershifted by anti-STAT-1 antibodies. The anti-STAT-5 antibodies had no effect. This indicates that STAT-3 and, to a lesser extent, STAT-1 are the major transcription factors activated by TIF. These results were also obtained using human hepatoma cell line HepG3, and also hepatoma cell line H4IIE.

### EXAMPLE 28

This example describes experiments designed to measure STAT activation by means of a reporter gene. The reporter was “pGRR5.” This contains 5 copies of the sequence set out in example 21, upstream of a luciferase gene under control of the TK promoter. The control was vector pRL-TK, which contains the remilla luciferase gene, under control of the TK promoter.

The assay was carried out by combining 106 HepG2 cells with 15 μg pGRR5, and 1 μg of Prl-TK (250V, 74 Ω, 1,200 μF). The pool of transfectants was divided into 24-well plates (42,000 cells/well).

After 1 hour, cells were stimulated with either human TIF (1% HEK293 cell supernatant) with 300 U/ml of human IL-6, 1% supernatant from mock transfected HEK293 cells, or medium alone.

After two hours, cells were pelleted, and lysed. Luciferase activity was monitored using standard methodologies. The results indicated that stimulation with the molecule of the invention increases transcriptional activity of a promoter that includes STAT-binding sites.

### EXAMPLE 29

Activation of STAT-3 by cytokines like IL-6 is known to result in acute phase protein induction in hepatoma cells. To determine if TIF exerted the same activity, 5×10⁶ HepG2 cells were stimulated for 2, 13, or 24 hours, with 1% supernatant from transiently infected HEK293-EBNA cells. In some experiments protein synthesis inhibitor cycloheximide was used at 10 μg/ml, and combined with the cells and supernatants. Following stimulation, total RNA was isolated using standard methodologies, and reverse transcription was performed on 10 μg samples of total RNA, using an oligo(dT) primer. Then, cDNA corresponding to 20 ng of RNA was amplified, for 18 cycles, with primers specific for human serum amyloid A (“SAA”), i.e.:

(sense, SEQ ID NO: 28)
agctcagcta cagcacagat
(antisense, SEQ ID NO: 29)
cctgccccat ttattggcag
Human α1 antichymotrypsin:
(sense, SEQ ID NO: 30)
tgtcctctgc caccctaaca
(antisense, SEQ ID NO: 31)
taattcacca ggaccatcat
for human haptoglobin:
(sense, SEQ ID NO: 32)
gtggactcag gcaatgatgt
(antisense, SEQ ID NO: 33)
acatagagtgt taaagtggg
and for human β-actin:
(sense, SEQ ID NO: 34)
gctggaaggt ggacagcgag
(antisense, SEQ ID NO: 35)
tggcatcgtg atggactccg.

For SAA, Tm was 54° C., while it was 52° C. for human α1-antichymotrypsin and haptoglobin, and 56° C. for β-actin. PCR products were analyzed, in ethidium bromide stained agarose gels, using standard methods.

The results indicated that TIF strongly induced SAA and α1-chymotrypsin and, to a lesser extent, haptoglobin. In order to determine if TIF directly upregulates SAA, or whether protein synthesis is required, HEPG2 cells were stimulated, as described, in the presence of cycloheximide. SAA expression was not affected, indicating that protein synthesis was not required for TIF activity.

### EXAMPLE 30

The results, supra, show that TIF and IL-6 appear to have similar activities on acute phase reactants. As IL-6 activity is mediated through gp 130, experiments were carried out to determine if TIF activity is mediated through gp 130 as well.

To determine if this was the case, HepG2 cells were transfected with a luciferase reporter, as described, supra, and were then stimulated with either TIF or IL-6, in the presence of polyclonal, anti-gp 130 antibodies which had been determined previously to block the activity of gp 130 interacting cytokines.

The results indicated that the polyclonals blocked only IL-6 activity.

Parallel experiments were carried out to determine if the IL-10R P chain was involved. In the presence of anti-IL-10R β antibodies, TIF activity was blocked completely while IL-6 activity was unaffected. The same affect was observed upon assaying for SAA expression.

### EXAMPLE 31

These experiments were designed to determine the ability of TIF to regulate acute phase proteins in vivo.

Varying amounts of recombinant murine TIF (50, 12.5, 3.2, 0.8 or 0.2 μg) were injected, intraperitoneally, into endotoxin resistant C3H/HeJ female mice (10-12 weeks old). Six hours after injection, the mice were killed, with the exception of the mice who received 50 μg of TIF, which were killed after 1, 3, 6, 12, or 24 hours. Livers were removed and directly frozen in liquid nitrogen. Total RNA was then extracted, using standard methodologies, after which 10 μg samples of total RNA were fractionated on a 1.3% agarose gel which contained 2.2 ml/liter formaldehyde. Samples were then transferred to nitrocellulose membranes.

Murine SAA probes were manufactured, using a commercial labelling kit. The hybridization assay was carried out in accordance with Van Snick, et al, J. Exp. Med 169:363-368 (1989), incorporated by reference. The probe itself was obtained via PCR, as described supra, using cDNA from murine liver, isolated as described supra. The primers used to manufacture the probe were:

| | |
|---|---|
| tctgctccct gctcctggga | (sense, SEQ ID NO: 36) |
| and | |
| tccaggaggt ctgtagtaat | (antisense, SEQ ID NO: 37) |

The results indicated that the highest dose of the murine TIF (50 μg), induced SAA expression in as little as 1 hour post i.p. injection. Maximal effect was reached after 6 hours. The expression level for SAA decreased at 24 hours past injection.

The data generated from the experiments using varying doses of TIF indicated that maximal induction of SAA still resulted when 3.2 μg dosages were used. SAA message was still detectable when as little as 0.8 μg were used.

### EXAMPLE 32

Initially, TIF was identified as a T cell derived cytokine. Most cytokines that regulate the liver acute phase are produced mainly during inflammation. In order to determine if TIF could be produced via inflammatory stimulation, in vivo, 2 μg of E. coli lipopolysaccharide antigen were injected, intraperitoneally into 12 week old, female BALB/c mice. Mice were killed two hours later, and organs were frozen in liquid nitrogen. Total RNA was isolated following standard protocols, and reverse transcription was performed on 10 μg total RNA, using oligo (dT) primers. Following the reverse transcription, cDNA corresponding to 20 ng of total RNA was amplified for 25 cycles, using primers specific for TIF, i.e.:

| | |
|---|---|
| ctgcctgctt ctcattgccc t | (sense, SEQ ID NO: 38) |
| and | |
| caagtctacc tctggtctca t, | (antisense, SEQ ID NO: 39) |

which had a Tm of 55° C. PCR products were analyzed via agarose gel electrophoresis.

The results indicated that LPS induced expression of TIF in all organs examined, indicating that TIF is involved in inflammatory processes.

The foregoing examples describe the invention, one aspect of which are isolated nucleic acid molecules, which encode TIF proteins such as those with the amino acid sequence of the protein encoded by the nucleotide sequence of SEQ ID NO: 7, 25 or 26. It will be appreciated by one of ordinary skill that the degeneracy of the genetic code facilitates the preparation of nucleic acid molecules which may not be identical to the nucleotide sequence of SEQ ID NO: 7, 25 or 26, but which encode the same protein. Of course, SEQ ID NOS: 7, 25 and 26 are preferred embodiments of this invention, but other embodiments are also a part of the invention. Genomic DNA, complementary DNA, and RNA, such as messenger RNA, are all to be included therein. Isolated nucleic acid molecules from other animal species, including other mammals, are also a part of the invention. A preferred aspect of the invention are isolated nucleic acid molecules whose complements hybridize to SEQ ID NO: 7, SEQ ID NO: 8, SEQ ID NO: 9, or SEQ ID NO: 25 or SEQ ID NO: 26 under stringent conditions. “Stringent conditions,” as used herein, refer, for example, to hybridization at 65° C. in buffer (3.5×SSC), 0.02% Ficoll, 0.02% polyvinylpyrrolidone, 0.02% bovine serum albumin, 25 mM NaH₂PO₄ (pH 7), 0.1% SDS, 2 mM EDTA, followed by a final wash at 2×SSC, room temperature and then 0.1×SSC/0.2×SDS at temperatures as high as, e.g., about 65° C. More stringent conditions, such as 0.1×SSC, can also be used. These nucleic acid molecules encode proteins of about 17-22 kD as determined by SDS-PAGE, which activates STAT proteins, such as STAT 1, STAT3 and/or STAT5. In glycosylated form, these proteins can range from about 17 to about 30 kilodaltons, as determined by SDS-PAGE. Also a port of the invention are isolated nucleic acid molecules which encode proteins having at least 30%, preferably at least 45%, more preferably at least 60%, and most preferably 90% amino acid identity with an amino acid sequence of a protein encoded by SEQ ID No: 7, 25 or 26, e.g., SEQ ID NO: 42.

Also a part of the invention are expression vectors which include the nucleic acid molecules of the invention, operably linked to a promoter, so as to facilitate expression of the DNA. It is well within the skill of the artisan to prepare such vectors.

The vectors, as well as the nucleic acid molecules per se, can be used to prepare recombinant cells, be these eukaryotic or prokaryotic, wherein either an expression vector or the nucleic acid molecule itself is incorporated therein. E. coli cells, COS cells, CHO cells, etc., are all examples of types of cells which may be used in accordance with this aspect of the invention.

Proteins encoded by the above referenced nucleic acid molecules, preferably in isolated form, are another feature of this invention. By “protein” is meant both the immediate product of expression of the nucleic acid molecules, glycosylated forms of it, as well as multimeric forms, such as dimers, trimers, and so forth. Also a part of the invention are multimers, such as dimers, which contain at least one protein molecule of the invention, and at least one, different protein molecule. Preferably, this different protein molecule is a cytokine, such as IL-10. Also included as a feature of the inventions are constructs, such as fusion proteins, where all or a part of the proteins described supra are linked in some fashion, such as in a fusion protein, to at least one additional protein or peptide, or amino acid sequence. The “fusion partner” may be, for example, a molecule which provides a recognizable signal, either directly or indirectly, such as a FLAG peptide, β-galactosidase, luciferase, and so forth. These fusion partners are preferably joined to the molecule which is described supra at the N- and/or C-terminus of the protein; however, it is to be understood that there are many techniques known for joining molecules to amino acids, and any and all of these methodologies can produce constructs which are a part of the invention.

The individual protein molecules of the invention, as noted supra, will preferably have a molecular weight of from about 17 to about 30 kilodaltons, as determined by SDS-PAGE. In multimeric forms, the molecular weight of the complex will, of course, vary, but the TIF molecules contained therein will each have a molecular weight of about 17 to 30 kilodaltons, as determined by SDS-PAGE.

The proteins preferably consist of at least about 120 and no more than about 200 amino acids. Preferably, the amino acids sequences consists of or comprises all or part of the amino acid sequences encoded by SEQ ID NOS: 7, 8, 9, 25 or 26. More preferably, the amino acid sequence contains all but about the first 40 amino acids encoded by said SEQ ID's. Even more preferably, it contains all but about the first 20 amino acids encoded by these sequences. Most preferably, the protein comprises amino acids set forth at SEQ ID NO: 40 or 41, as well as proteins defined by the amino acid identities set out supra.

It will be appreciated by the skilled artisan that the proteins encoded by the above recited nucleic acid molecules are a feature of the invention, and may be used to produce antibodies, in accordance with standard protocols. Such antibodies, in monoclonal and polyclonal form, constitute a further feature of the invention as do fragments of said antibodies, chimeric forms, humanized forms, recombinant forms, and so forth. Also a feature of the invention are immunogens, comprising all or a part of the amino acid sequence protein molecules of the invention, preferably combined with an adjuvant, such as Complete or Incomplete Freund's Adjuvant. Portions of the protein sequences may be linked to other molecules, such as keyhole limpet hemocyanin, to render them more immunogenic. These antibodies can be used, e.g., to determine if the proteins of the invention are present. This is a further feature of the invention, as is now explained. It has been shown, in the examples, that the nucleic acid molecules of the invention were expressed in the presence of the IL-9. Hence, a further feature of the invention is a method to determine if IL-9 is or has been present, wherein one detects either the proteins of the invention, using antibodies for example, or mRNA using the nucleic acid molecules of the invention, as probes. The mRNA can be determined directly, or in the form of cDNA. Such probes may or may not be labeled, as a matter of choice for the user. Hence, one can determine, for example, if, following administration of IL-9, the cytokine is still efficacious, by determining if the nucleic acid molecule of the invention is present. This type of assay can be adapted, for quantitative studies, wherein one determines, for example, either if a cell is sensitive to IL-9, and if so, how sensitive it is. One can also use the proteins of the invention to phosphorylate STAT proteins such as STAT1, STAT3 and/or STAT 5. This in turn results in dimerization of the STAT protein, followed by migration to the nucleus to provoke the effect that these STAT proteins have on cells.

One could also use these molecules to test the efficacy of IL-9 agonists or antagonists when administered to a subject, such as a subject suffering from lymphoma, an immune system disorder such as an allergy, acquired immune deficiency syndrome, autoimmune diabetes, thyroiditis, or any of the other conditions described in, e.g, U.S. Pat. Nos. 5,830,454; 5,824,551, and pending application Ser. No. 08/925,348, filed on Sep. 8, 1997 now allowed, all of which are incorporated by reference. The molecules can also be used to mediate the role of IL-9 in these and other conditions. To elaborate, since IL-9 induces TIFs, the TIFs are useful as IL-9 activity mediators. Thus, a further aspect of the invention is a method to determine activity of endogenous IL-9, such as in situations where excess IL-9 activity is implicated, such as asthmas, allergies, and lymphomas. One can also block or inhibit IL-9 activity by blocking or inhibiting TIF or TIF activity, using, e.g., antisense molecules, antibodies which bind to TIF, or other antagonists of these molecules. For example, muteins of TIF, which bind to the TIF receptor but do not activate it, thereby inhibiting IL-9 induced activity, are a feature of the invention. Examples of conditions which can be treated by the use of such TIF muteins are allergies, asthma, and so forth. Muteins in accordance with the invention can be made in accordance with, e.g., Weigel, et al, Eur. J. Biochem 180(2):295-300(1989) and Epps, et al, Cytokine 9(3): 149-156(1997), both of which are incorporated by reference. Such muteins can be used in the treatment of asthma, allergies, or both. Further, it will be clear to the skilled artisan that the models set forth, supra, can also be used to screen for appropriate muteins. The ability to regulate IL-9 activity is important in conditions such as those listed supra, as well as conditions such as apoptosis, including cortisol induced apoptosis, conditions involving the nuclear expression of BCL-3, since IL-9 is known to induce such expression, and so forth. “Antibodies,” as used herein, refers to any portion of an antibody which binds to TIF, including chimeric and humanized antibodies.

Another feature of the invention relates to the ability of the TIF type molecules of the invention to either promote regeneration or inhibit differentiation of tissue types on which the molecules are active. As was shown, supra, the TIF molecules target various cancer and normal cell lines (i.e., mesangial and neuronal cells, as well as melanoma and hepatoma cells). Hence, one can stimulate regeneration of tissue via, e.g., adding an amount of a TIF type molecule to a sample in need of regeneration of a tissue acted on by the TIF molecule. This approach can be used both in vitro, and in vivo. Similarly, antagonists of TIF may be added when the situation is one where the aim is to inhibit differentiation of a particular type of tissue, such as melanoma or hepatoma.

The genes which encode TIF, as noted in Example 25, supra, are located on chromosome 12. This chromosome is associated with asthma, as is known in the art. It is also known that region 12q15 is associated with other inflammatory disease. Hence, a further embodiment of the invention is a method for determining susceptibility to conditions such as, or related to asthma, by determining if aberrations, such as polymorphisms, deletions, additions, etc., are present at the site of the TIF gene. Such aberrations may be an indicia of susceptibility to, or of the presence of, asthma, an allergic condition, or one or more related conditions. The ability to detect aberrations in a DNA sequence is well known in the art, and such methods need not be set forth herein. Preferably, the aberration or aberrations is detected via standard techniques, such as PCR, using the methodologies and primers referred to supra.

The data set forth supra, in particular the induction of expression of the molecule of the invention by LPS, and the regulation of the acute phase response by this molecule, indicate that the molecules of the invention are actively involved in the inflammatory response. Hence a further feature of this invention relates to identification of proinflammatory and anti-inflammatory agents via use of IL-TIF/IL-21. It will be clear to the skilled artisan that IL-TIF/IL-21 can regulate the inflammatory response. A preferred aspect of this regulation is the modulation of the acute phase response by organs, such as the liver, by administering either IL-TIF/IL-21, or an antagonist thereof. See, e.g., Janeway, et al., Immunobiology (4^{th} edition), incorporated by reference. Janeway explains that various cytokines such as IL-1, IL-6 and TNF-α activate hepatocytes to synthesize acute phase proteins, such as c-reactive protein, and mannan binding lectin, as well as those described in the examples, supra.

The role of IL-TIF/IL-21 in activating acute phase proteins also leads to a method for identifying agonists and antagonists of IL-TIF/IL-21, by determining if acute phase protein production changes in the presence of the putative agonist or antagonist, and IL-TIF/IL-21. An increase in production should be taken as an indicia that the test molecule is an agonist, and vice versa.

Also a part of the invention are methods for regulating activity of IL-TIF/IL-21 in view of its relationships to interleukin-10 receptors. As was shown, supra, the use of IL-10Rβ antagonists, such as antibodies, inhibit IL-TIF/IL-21 activity. Hence, another feature of the invention is the use of agonists and antagonists of IL-10 receptors, such as IL-10Rβ agonists, and antagonists, to regulate IL-TIF/IL-21 production.

Other features of the invention will be clear to the artisan and need not be discussed further.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

1. A method for stimulating expression of a STAT transcription factor, comprising contacting a cell capable of said expression with an amount of an IL-TIF/IL-21 to said cell sufficient to stimulate said expression.

2. The method of claim 1, wherein said STAT transcription factor is STAT 3 or STAT 1.

3. The method of claim 2, wherein said STAT transcription factor is STAT 3.

4. The method of claim 1, wherein said IL-TIF/IL-21 is a mammalian IL-TIF/IL-21.

5. The method of claim 4, wherein said mammalian IL-TIF/IL-21, IL-TIF/IL-21 is human IL-TIF/IL21.

6. The method of claim 5, wherein said human IL-TIF/IL-21 is encoded by SEQ ID NO: 25 or SEQ ID NO: 26.

7. A method for inducing production of an acute phase protein in a cell, comprising contacting said cell with an amount of an IL-TIF/IL-21 sufficient to induce production of said acute phase protein.

8. The method of claim 7, wherein said cell is a liver cell.

9. The method of claim 7, wherein said acute phase protein is human serum amyloid A, α1 chymotrypsin, or haptoglobin.

10. The method of claim 7, wherein said IL-TIF/IL-21 is a mammalian IL-TIF/IL-21.

11. The method of claim 10, wherein said mammalian IL-TIF/IL-21 is human IL-TIF/IL-21.

12. The method of claim 11, wherein said human IL-TIF/IL-21 is encoded by SEQ ID NO: 25 or SEQ ID NO: 26.

13. A method for modulating activity of an IL-TIF/IL-21 molecule, comprising contacting a cell susceptible to IL-TIF/IL-21 activity with an IL-TIF-IL-21 modulator, in an amount sufficient to modulate IL-TIF/IL-21 activity.

14. The method of claim 13, wherein said modulator is a substance which binds to IL-10Rβ molecules.

15. The method of claim 14, wherein said modulator is an antibody which binds specifically to an IL-10Rβ molecule.
